(19) United States
(12) Patent Application Publication
(10) Pub. No.: US 2008/0124753 A1
(43) Pub. Date: May 29, 2008

(54) SpiceMatrix Technology for Taste Compound Identification

(76) Inventors: Seunghun Paul LEE, Newtown, PA (US); M.N. Tulu BUBER, Newtown, NJ (US); Ivona BAKAJ, Cranbury, NJ (US); Robert W. BRYANT, Princeton, NJ (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/940,752

(22) Filed: Nov. 15, 2007

Related U.S. Application Data
(60) Provisional application No. 60/858,938, filed on Nov. 15, 2006.

(51) Int. Cl. C12Q 1/02

(52) U.S. Cl. 435/29

(57) ABSTRACT

The present invention is related to a screening method to identify compounds that impact taste. Reactivity profiles of spice compounds are determined by assaying activity in test cells expressing various ion channels. The reactivity profiles can be used to identify novel taste compounds having similar taste effects.

### CROSS-REFERENCE TO RELATED APPLICATIONS

This application claims the benefit of U.S. Provisional Application No. 60/858,938, filed Nov. 15, 2006, which is herein incorporated by reference.

### BACKGROUND OF THE INVENTION

1. Field of the Invention

The present invention is related to a screening method to identify compounds that impact taste. More specifically, the present invention relates to a screening method useful in the generation of a taste profile for compounds that affect taste sensation by modulating the activity of certain ion channels. The present invention also provides for the ability to screen for tastants with similar taste properties. By comparing the activity of tastants on the ion channels to the activity of known tastants, putative tastants of the same type can be identified or differentiated.

2. Background

Taste perception not only plays a critical role in the nutritional status of human beings, but is also essential for the survival of both lower and higher animals (Margolskee, R. F. J. Biol. Chem. 277:1-4 (2002); Avenet, P. and Lindemann, B. J. Membrane Biol. 112:1-8 (1989)). The ability to taste has significance beyond providing people with pleasurable culinary experiences. For example, the ability to taste allows us to identify tainted or spoiled foods, and provides satisfying responses that may be proportionate to caloric or nutritive value.

Although taste perception is a vital function, sometimes it is useful to modify certain tastes. For example, many active ingredients in medicines produce undesirable tastes, such as a bitter taste or a pungent burning sensation. Inhibition of this bitter taste or burning sensation could lead to improved acceptance by the patient. In other circumstances, it may be desirable to enhance the unpleasant taste of something that would be toxic if ingested.

The effects of many compounds on taste is well known. For instance, capsaicin is associated with the sensation of heat upon ingestion of chili peppers, while gingerol is associated with the “hot” sensation of ginger.

Ion channels are transmembrane proteins that form pores in a membrane and allow ions to pass from one side to the other (reviewed in B. Hille (Ed), 1992, Ionic Channels of Excitable Membranes 2nd ed., Sinauer, Sunderland, Mass.). Several ion channels have been shown to be essential for taste transduction (Perez et al., Nature Neuroscience 5:1169-1176 (2002); Zhang et al., Cell 112:293-301 (2003)). The effects that well known taste compounds have on ion channel activity have also begun to be analyzed. For example, menthol has been shown to activate the TRPM8 (Behrendt, H.-J., et al., Brit. J. Pharm. 141:737-745 (2004)); while garlic has been shown to activate TRPA1 (Bautista, D. M. et al. Proc. Natl. Acad. Sci. USA 102:12248-12252 (2005)).

Therefore, there exists a need in the art to provide a method to rapidly screen compounds and select those having a taste modifying ability. The use of a molecular-based taste profile, or SpiceMatrix, can provide a selective method to evaluate the molecular effects of complex spices by dissecting their effects into individual components. The SpiceMatrix can also provide the basis for the ability to predict the taste modifying ability of unknown compounds.

### BRIEF SUMMARY OF THE INVENTION

A new screening assay has been discovered that allows for the rapid generation of a taste profile for taste modifying compounds. The method of the invention relies on the generation of a taste profile, or SpiceMatrix, of taste modifiers on a panel of ion channels. The reactivity pattern can be used as a predictor of the effects of candidate compounds on taste. The method will allow thousands of compounds that potentially modulate ion channels, and affect taste, to be screened quickly and reliably, as well as assessed for novelty.

An embodiment of the invention is a method for generating a taste profile for compounds comprising: (a) contacting said compound with at least two groups of isolated test cells expressing a transient receptor potential (TRP) ion channel, wherein each group of test cells expresses a different recombinant TRP ion channel; (b) measuring the activity of the test cells of step (a) in the presence of the spice compound; (c) comparing the measured activity in step (b) to the activity of the test cells which do not express a TRP ion channel in the presence of the compound to determine the extent of TRP modulation; and (d) generating an activity profile of the at least two TRP ion channels.

In some embodiments, the two or more TRP ion channels are selected from TRPA1, TRPV1, TRPV3, TRPM8 and TRPM5. In additional embodiments, three or more TRP ion channels are analyzed. In further embodiments, four or more TRP ion channels are analyzed.

In some embodiments, the activity is determined by measuring the fluorescent intensity of the cell. In a further embodiment, the activity is determined in a high throughput assay.

In additional embodiments, the claimed method is directed to screening cells that are located in a multi-well vessel. The multi-well vessels of the claimed invention may contain up to and a number equaling 96 wells. In another embodiment, the multi-well vessel comprises greater than 96 wells. In another embodiment, the multi-well vessel comprises 384 wells. In yet another embodiment, the multi-well vessel comprises 1536 wells.

In some embodiments, the test cells of the claimed method are HEK-293, Hela, Chinese Hamster Ovary or COS cells.

In some embodiments of the claimed method, the fluorescent intensity is measured using a membrane potential fluorescent dye. In additional embodiments, the membrane potential fluorescent dye is a Fluorescent Imaging Plate Reader Membrane Potential (FMP) dye. In another embodiment, the fluorescent intensity is measured using a calcium dye.

In some embodiments of the claimed method, the fluorescent intensity is measured using an optical detector. In additional embodiments, the optical detector is selected from a Fluorescent Imaging Plate Reader (FLIPR®), FLEXStation, Voltage/Ion Probe Reader (VIPR), fluorescent microscope and charge-coupled device (CCD) camera or Pathway HT.

The invention also relates to a method of manipulating the taste profile of a compound comprising: (a) contacting said compound with at least two groups of isolated test cells expressing a TRP ion channel, wherein each group of test cells expresses a different recombinant TRP ion channel; (b) measuring the activity of the test cells of step (a) in the presence of the compound; (c) comparing the measured activity in step (b) to the activity of the test cells which do not express a TRP ion channel in the presence of the compound to determine the extent of TRP modulation; (d) generating a reactivity profile of the at least two TRP ion channels; and (e) altering the reactivity of said compound with said TRP ion channels.

The invention also relates to a method for identifying novel taste compounds comprising: (a) determining the reactivity of known taste compounds to at least two groups of isolated test cells expressing a TRP ion channel, wherein the groups of test cells express a different recombinant TRP ion channel; (b) contacting at least two different groups of isolated test cells expressing a TRP ion channel with a potential taste compound, wherein the test cells express the same recombinant TRP ion channels as in step (a); (c) measuring the activity of the test cells of step (b) in the presence of the potential taste compound; (d) comparing the measured activity to the activity of test cells that do not express a TRP ion channel to determine the extent of TRP modulation; (e) comparing the reactivity of known taste compounds of step (a) to the reactivity of the potential taste compound to the at least two different recombinant TRP ion channels; and (f) selecting one or more taste compounds that display a similar TRP ion channel reactivity pattern to known taste compounds.

Further embodiments, features, and advantages of the present inventions, as well as the structure and operation of the various embodiments of the present invention, are described in detail below with reference to the accompanying drawings.

### BRIEF DESCRIPTION OF THE DRAWINGS/FIGURES

The accompanying drawings, which are incorporated herein and form a part of the specification, illustrate one or more embodiments of the present invention and, together with the description, further serve to explain the principles of the invention and to enable a person skilled in the pertinent art to make and use the invention.

FIG. 1 shows the effects of individual compounds on various ion channels as measured by a change in relative fluorescent intensity of a voltage-sensitive dye in the cell. (A) cinnamaldehyde activates TRPA1; (B) carbachol increases TRPM5; (C) capsaicin activates TRPV1; and (D) menthol activates TRPM8.

FIGS. 2A-2C show the relative stimulation of 68 different compounds on TRPA1, TRPV1, TRPM8 and TRPM5 relative to untransfected HEK-293 cells (Parentals) (FIG. 2A) along with the dose response and profile pattern of each compound (FIGS. 2B-2C).

FIG. 3 shows the reactivity profile for a 23-member subset of the 68 compounds shown in FIG. 2 on the TRPA1, TRPV1, TRPM8 and TRPM5 ion channels.

### DETAILED DESCRIPTION OF THE INVENTION

### Overview

The invention is a screening assay for identification of compounds that affect taste. The effect that many compounds, such as cinnamaldehyde and capsaicin, have on taste are well known. Since the effect those known taste compounds have on ion channel activity can be measured, a reactivity profile, or “SpiceMatrix” of relative ion channel activity can be developed for those compounds. The SpiceMatrix can then be used as a comparative tool to identify candidate compounds that will have similar taste properties to the known compounds. In this way, the activity of known compounds can act as a predictor of taste effects of candidate compounds.

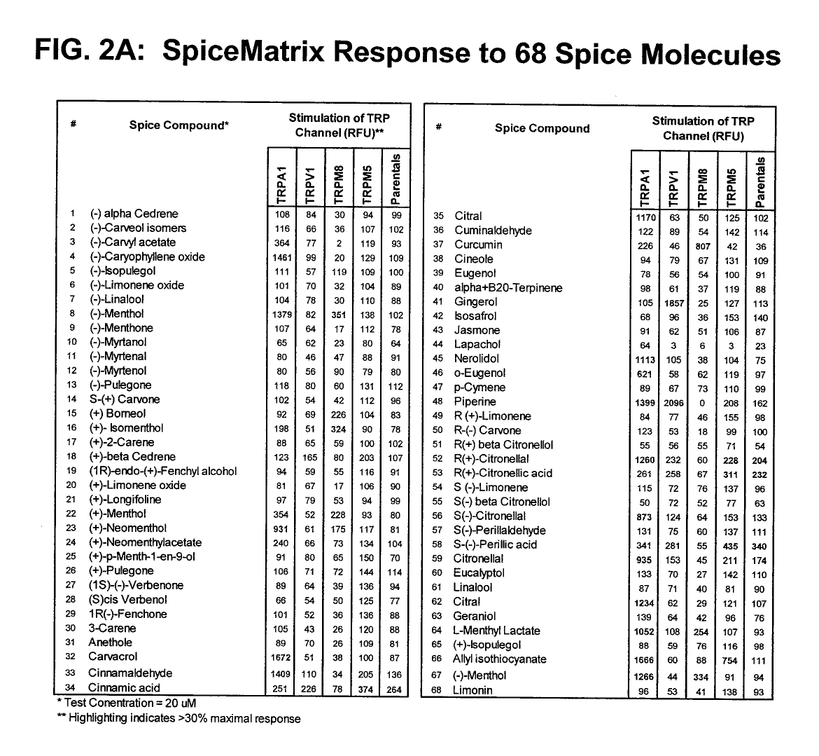

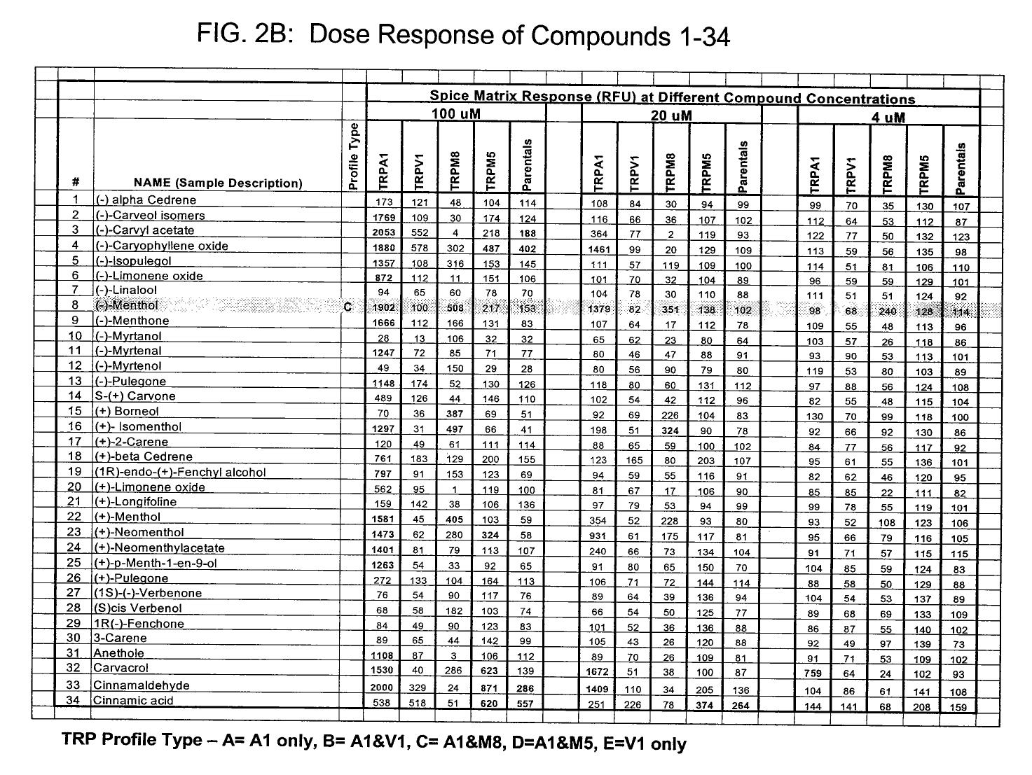

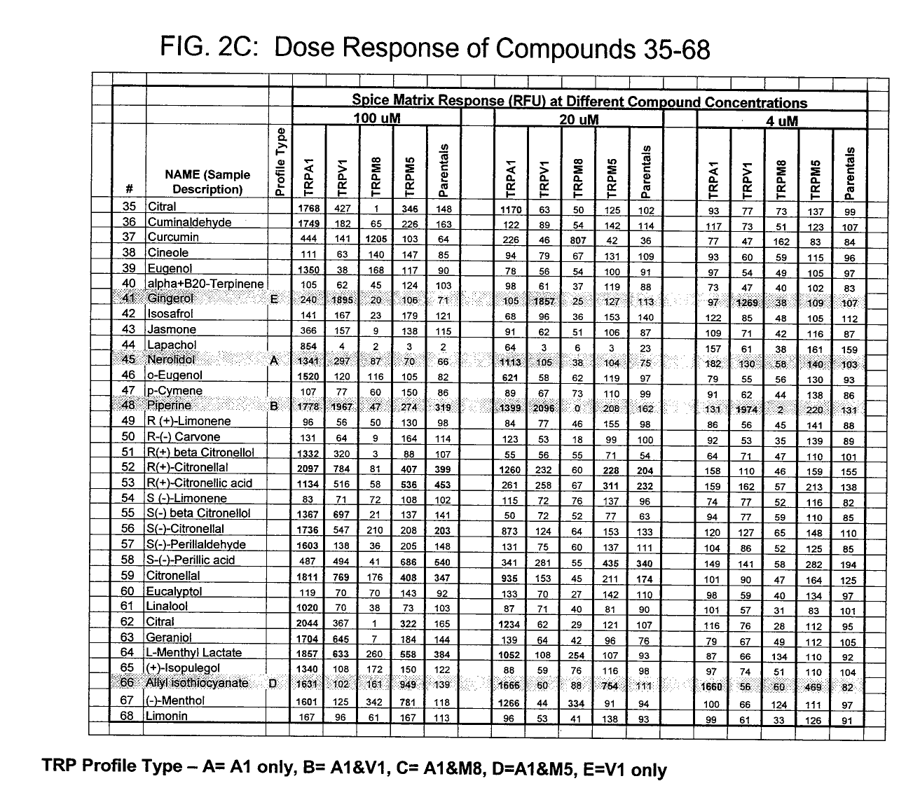

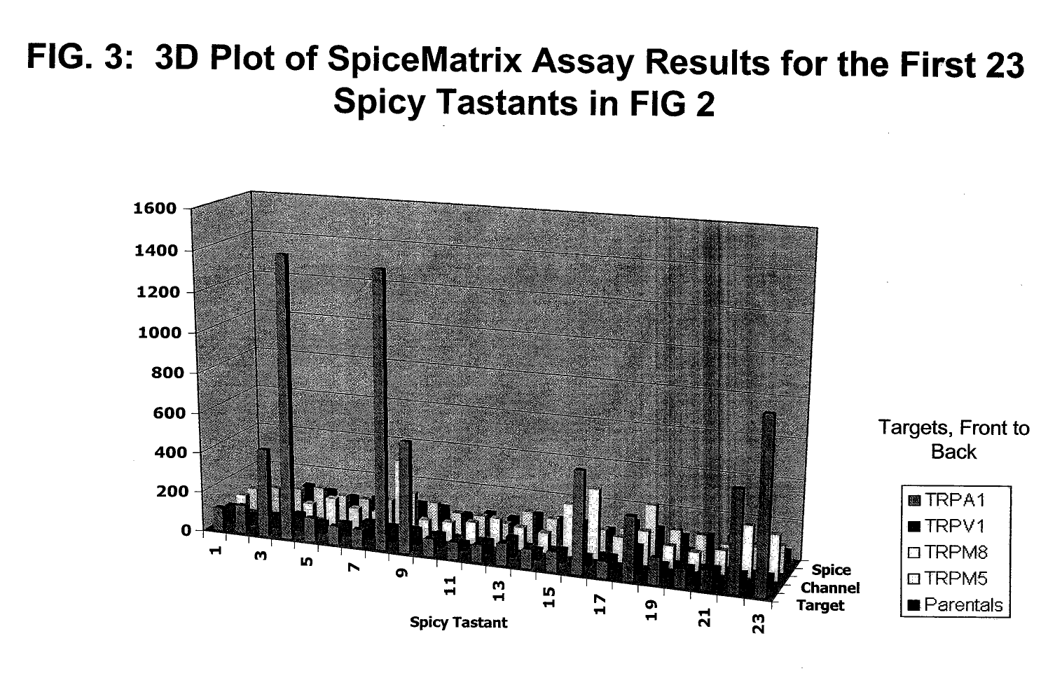

As used herein, a “reactivity profile” is an activity pattern for a compound when assayed for ion channel activity at a given concentration. For example, in a four ion channel screen, compound 1 may be reactive with channels 1 and 4, but not 2 and 3; while compound 2 may be reactive with channels 2, 3 and 4, but not channel 1. (See, e.g. FIG. 2A). The profiles are validated with dose response studies (See, e.g. FIGS. 2B-2C).

Taste is the ability to respond to dissolved molecules and ions called tastants. Humans detect taste with taste receptor cells (TRCs), which are clustered in taste buds. (Kinnamon, S. C. TINS 11:491-496 (1988)). Tastants bind specific receptors on the TRC's cell membrane, leading to a voltage change across the cell membrane. A change in voltage across the TRC cell membrane depolarizes, or changes the electric potential of the cell. This leads to a signal being sent to a sensory neuron leading back to the brain.

Taste however is not limited to sensations that are detected by TRCs. Tastes are generally made up of a variety of components such as odor and hot/cold sensations. A clear example of this is the taste associated with hot pepper, or capsaicin. Therefore, the reactivity profile described herein, can also be applied to compounds that affect odor and hot/cold sensations.

Ion channels have “gates” that open in response to a specific stimulus. As examples, voltage-gated channels respond to a change in the electric potential across the membrane, mechanically-gated channels respond to mechanical stimulation of the membrane, and ligand-gated channels respond to the binding of specific molecules. Various ligand-gated channels can open in response to extracellular factors, such as a neurotransmitters (transmitter-gated channels), or intracellular factors, such as ions (ion-gated channels), or nucleotides (nucleotide-gated channels). Still other ion channels are modulated by interactions with other proteins, such as G-proteins (G-protein coupled receptors or GPCRs).

Most ion channels mediate the permeation of one predominant ionic species. For example, sodium (Na⁺), potassium (K⁺), chloride (Cl⁻), and calcium (Ca²⁺) channels have been identified.

The transient receptor potential (TRP) family ion channels have been implicated in the mechanisms controlling several relevant physiological responses, including temperature and mechanical stimulation, responses to painful stimuli, taste, and pheromones (Calixto, J. B. et al., Pharmacology and Therapeutics 106:179-208 (2005)). The TRP family of ion channels has been subdivided into four main classes: TRPC (short cannonical TRP channels); TRPM (long, TRP melastatin channels); TRPV (vanilloid receptor TRP channels); and TRPA (short ankyrin-repeat TRP channels) (Clapham, D. E. et al., Pharmacol. Rev. 55:591-596 (2003)).

One member of the TRPA family, TRPA1, has been shown to be sensitive to low temperatures, with activation of the channel occurring at an average temperature of about 18° C. (about 64° F.). (Story, G. M. et al., Cell 112: 819-829 (2003)). TRPA1 channels are also activated by naturally occurring substances such as isothiocyanate compounds, A⁹-tetrahydrocannabinol (THC), and cinnamaldehyde. (Jordt, S. E. et al., Nature 427: 260-265 (2004); Bandell, M. et al., Neuron 41: 849-857 (2004)). In addition, mouse TRPA1-CHO cells show a sharp increase in intracellular free Ca²⁺ upon application of several plant derived compounds such as eugenol (from clove oil), gingerol (from ginger) and methyl salicylate (from wintergreen oil). (Blandell, M. et al.) Allyl isothiocyanate, cinnamaldehyde, eugenol, gingerol and methyl salicylate cause a pungent burning sensation in humans, e.g., cinnamaldehyde is a key component responsible for cinnamon flavor.

TRPV1 is a receptor-activated non-selective calcium permeant cation channel involved in detection of noxious chemical and thermal stimuli. TRPV1 may also be involved in mediation of inflammatory pain and hyperalgesia. TRPV1 is activated by vanilloids, like capsaicin, and temperatures higher than 42° C. and exhibits a time- and Ca⁺²-dependent outward ion flux. TRPV1 can be activated by endogenous compounds, including 12-hydroperoxytetraenoic acid, and endocannabinoids, like anandamide and bradykinin.

TRPV3 is believed to belong to a family of nonselective cation channels that function in a variety of processes, including temperature sensation and vasoregulation. The thermosensitive members of this family are expressed in subsets of sensory neurons that terminate in the skin, and are activated at distinct physiological temperatures. This channel is activated at temperatures between 22 and 40° C. This gene lies in close proximity to TRPV1 on chromosome 17, and the two encoded proteins are thought to associate with each other to form heteromeric channels. (See, Smith, G. D. et al., Nature 418:186-190 (2002); Xu, H. et al., Nature 418:181-186 (2002)).

TRPM5 is believed to be activated by stimulation of a receptor pathway coupled to phospholipase C and by IP₃-mediated Ca²⁺ release. The opening of this channel is dependent on a rise in Ca²⁺ levels (Hofmann et al., Current Biol. 13:1153-1158 (2003)). TRPM5 is also a necessary part of the taste-perception machinery and has been shown to play a role in bitter, sweet and umami taste (Talayera, K. et al., Nature 438:1022-1025 (2005)).

TRPM8 is also considered a “cold” receptor similar to TRPA1. TRPM8 is specifically expressed in a subset of pain- and temperature-sensing neurons (Peier, A. M. et al., Cell 108:705-15 (2002)). Cells overexpressing the TRPM8 channel can be activated by cold temperatures and by the cooling agent, menthol (McKemy, D. D. et al., Nature 416:52-58 (2002)). TRPM8 is also upregulated on a variety of primary tumors (Alexander, S. P. H. et al., Brit. J. Pharmacol. 147:S3 (2006)).

Although taste perception is a vital function, the inhibition, or masking, of undesirable tastes is beneficial under certain circumstances. For example, many active pharmaceutical ingredients of medicines produce undesirable tastes, such as a bitter taste. Inhibition of the bitter taste produced by the medicine may lead to improved acceptance by the patient. In other circumstances, enhancement of taste may be desirable as in the case of developing improved artificial sweeteners or in treatment of taste losses in groups such as the elderly (Mojet et al., Chem Senses 26:845-60 (2001)).

Eugenol, gingerol and methyl salicylate have been shown to activate TRPV1 and TRPM8 in addition to TRPA1 and thus, produce their pungent activity through the stimulation of a variety of TRP ion channels. (Calixto et al.). In contrast, allyl isothiocyanate and cinnamaldehyde are specific activators of TRPA1. TRPA1 may be responsible for the burning taste sensory quality of allyl isothiocyanate and cinnamaldehyde.

As used in the specification and claims, the singular form “a”, “an” and “the” include plural references unless the context clearly dictates otherwise. For example, the term “an ion channel” includes a plurality of ion channels. The term “a cell” includes a plurality of cells.

Groups of cells expressing various ion channels are exposed to compounds and the ability of those compounds to stimulate opening or to block opening of the ion channels is measured. A reactivity pattern, or SpiceMatrix is then provided for each compound. A reactivity pattern is also generated using cells which do not express the ion channels. By comparing the two patterns, the degree of ion channel modulation can be ascertained. The modulation of the ion channels creates the reactivity profile. The reactivity profile is used to identify compounds having a desired taste profile. A fluorescent dye that responds to changes in cell membrane potential may be used for detection.

Once the reactivity pattern for a compound is identified, it can be used to alter the “taste” associated with the compound. “Taste,” as used herein, includes not only sensations detected by TRCs, but also odor and hot/cold temperature sensations. By altering the reactivity pattern of the compounds to more closely mimic the reactivity of other known compounds, the taste perception of the compound can be altered. For example, as shown in FIG. 2, allyl isothiocyanate (wasabi) is highly reactive with the ion channels TRPA1 and TRPM5. The taste associated with a test compound could be altered to more closely resemble wasabi taste perception by altering the test compound's SpiceMatrix to resemble that of wasabi. As stated above, it is appreciated that since many factors contribute to taste, altering a compound's SpiceMatrix will not produce an identically perceived compound, but rather more closely mimic a given perception.

While specific configurations and arrangements are discussed, it should be understood that this is done for illustrative purposes only. A person skilled in the pertinent art will recognize that other configurations and arrangements can be used without departing from the spirit and scope of the present invention. It will be apparent to a person skilled in the pertinent art that this invention can also be employed in a variety of other applications.

### Cells

Cells for use in the method of the invention contain functional ion channels. The ion channels of the invention include, but are not limited to, TRPA1, TRPV1, TRPV3, TRPM8 and TRPM5 (“the ion channels”). The practitioner may use cells in which the ion channels are endogenous or may introduce the ion channels into a cell. If ion channels are endogenous to the cell, but the level of expression is not optimum, the practitioner may increase the level of expression of the ion channels in the cell. Where a given cell does not produce the ion channels at all, or at sufficient levels, a nucleic acid encoding the ion channels may be introduced into a host cell for expression and insertion into the cell membrane. The introduction, which may be generally referred to without limitation as “transformation,” may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. General aspects of mammalian cell host system transformations have been described in U.S. Pat. No. 4,399,216. For various techniques for transforming mammalian cells, see Keown et al., Meth. Enzym., 185:527-537 (1990) and Mansour et al., Nature 336:348-352 (1988).

TRPM5 (also known as TRP8, LTRPC5, MTR1 and 9430099A1Rik) is expressed as a 4.5 kb transcript in a variety of fetal and adult tissues (Prawitt et al. Hum. Mol. Gen. 9:203-216 (2000)). Human TRPM5 has a putative reading frame containing 24 exons which encode an 1165 amino acid, membrane spanning polypeptide. The National Center for Biotechnology Information (NCBI) database lists several sequences for both the nucleic acid (NP_{—}064673, NP_{—}055370, AAP44477, AAP44476) and amino acid (NM_{—}014555, NM_{—}020277, AY280364, AY280365) sequences for both the human and mouse forms of TRPM5, respectively. The inclusion of the above sequences is for the purpose of illustration of the TRPM5 genetic sequence, however the invention is not limited to one of the disclosed sequences.

TRPM8 (also known as TRPP8, LTRPC6, MGC2849, CMR1, Trp-p8 and MGC2849) is expressed as a 5.6 kb transcript in a variety of human tissues (Tsavaler, L. et al., Cancer Res. 61:3760-3769 (2001)). Human TRPM8 has a putative reading frame containing seven transmembrane domains encoded by an 1104 amino acid. The NCBI database lists several sequences for both the nucleic acid (AB061779, AY090109, AY328400, AY532375, AY532376, BC001135, BC033137 and DQ139309) and amino acid (BAB86335, AAM10446, AAP92167, AAS45275, AAH01135 and AAZ73614) sequences for many forms of TRPM8. The inclusion of the above sequences is for the purpose of illustration of the TRPM8 genetic sequence, however the invention is not limited to one of the disclosed sequences.

TRPA1 (also known as p120, ANKTM1, CG5751, dTRPA1 and dANKTM1) is expressed as a 4.2 kb transcript in human tissues (Jaquemar, D., et al., J. Biol. Chem. 274:7325-7333 (1999)). The open reading frame of the mRNA encodes a protein of 1119 amino acids forming two distinct domains. The amino-terminal domain consists of 18 repeats that are related to the cytoskeletal protein ankyrin. The carboxy-terminal domain contains six putative transmembrane segments that resemble many ion channels. The NCBI database lists several sequences for both the nucleic acid (Y10601, AE003554, AY496961, AK045771 and AY231177) and amino acid (CAA71610, AAF50356, AAS78661, BAC32487 and AAO43183) sequences for many forms of TRPA1. The inclusion of the above sequences is for the purpose of illustration of the TRPA1 genetic sequence, however the invention is not limited to one of the disclosed sequences.

TRPV1 (also known as VR1, DKFZp434K0220, VR-1 and OTRPC1) is expressed as a 4.0 kb transcript in human tissues (Caterina, M. J., et al., Nature 389:816-824 (1997)). The open reading frame of the mRNA encodes a protein of 839 amino acids. The NCBI database lists several sequences for both the nucleic acid (NM_{—}018727, AF196175, AF196176, AF235160, AJ272063, AJ277028, AL136801, AY131289, AY986821, DQ177332 and DQ177333) and amino acid (AAG43466, AAG43467, AAN73432, CAB89866, CAB95729, CAB66735, AAM89472, AAX84657, ABA06605 and ABA06606) sequences for many forms of TRPV1. The inclusion of the above sequences is for the purpose of illustration of the TRPV1 genetic sequence, however the invention is not limited to one of the disclosed sequences.

TRPV3 (also known as transient receptor potential cation channel, subfamily V, member 3; vanilloid receptor 3 or vanilloid receptor-related osmotically activated channel protein) is expressed as a 3.4 kb transcript in human tissues. The open reading frame of the mRNA encodes a protein of 790 amino acids. The NCBI database lists several sequences for both the nucleic acid (AF514998.1, AJ487035.2, AK074032.1, AK127726.1, AY118268.1, BC104866.1, BC104868.1 and BX537539.1) and amino acid (AAM54027.1, CAD31711.2, BAB84858.1, AAM80558.1, AAM80559.1, AAI04867.1 and AAI04869.1) sequences for many forms of TRPV3. The inclusion of the above sequences is for the purpose of illustration of the TRPV3 genetic sequence, however the invention is not limited to one of the disclosed sequences.

It is recognized in the art that there can be significant heterogeneity in a gene sequence depending on the source of the isolated sequence. The invention contemplates the use of conservatively modified variants of the ion channels. Conservatively modified variants applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence to essentially identical sequences. Because of the degeneracy of the genetic code, a large number of functionally identical nucleic acids encode any given protein.

For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein, which encodes a polypeptide, also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid, which encodes a polypeptide, is implicit in each described sequence.

Conservative substitution tables providing functionally similar amino acids are well known in the art. For example, one exemplary guideline to select conservative substitutions includes (original residue followed by exemplary substitution): ala/gly or ser; arg/lys; asn/gln or his; asp/glu; cys/ser; gln/asn; gly/asp; gly/ala or pro; his/asn or gln; ile/leu or val; leu/ile or val; lys/arg or gln or glu; met/leu or tyr or ile; phe/met or leu or tyr; ser/thr; thr/ser; trp/tyr; tyr/trp or phe; val/ile or leu. An alternative exemplary guideline uses the following six groups, each containing amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (I); 5) Isoleucine (I), Leucine (L), Methionine (M), Valine (V); and 6) Phenylalanine (F), Tyrosine (Y), Tryptophan (W); (see also, e.g., Creighton, Proteins, W. H. Freeman and Company (1984); Schultz and Schimer, Principles of Protein Structure, Springer-Verlag (1979)). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in an encoded sequence can also be considered “conservatively modified variations.”

The variant ion channel proteins of the invention comprise non-conservative modifications (e.g. substitutions). By “nonconservative” modification herein is meant a modification in which the wildtype residue and the mutant residue differ significantly in one or more physical properties, including hydrophobicity, charge, size, and shape. For example, modifications from a polar residue to a nonpolar residue or vice-versa, modifications from positively charged residues to negatively charged residues or vice versa, and modifications from large residues to small residues or vice versa are nonconservative modifications. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine. In one embodiment, the variant ion channel proteins of the present invention have at least one nonconservative modification.

The variant proteins may be generated, for example, by using a PDA™ system previously described in U.S. Pat. Nos. 6,188,965; 6,296,312; 6,403,312; alanine scanning (see U.S. Pat. No. 5,506,107), gene shuffling (WO 01/25277), site saturation mutagenesis, mean field, sequence homology, polymerase chain reaction (PCR) or other methods known to those of skill in the art that guide the selection of point or deletion mutation sites and types.

The cells used in methods of the present invention may be present in, or extracted from, organisms, may be cells or cell lines transiently or permanently transfected or transformed with the appropriate proteins or nucleic acids encoding them, or may be cells or cell lines that express the required ion channels from endogenous (i.e. not artificially introduced) genes.

Expression of the ion channel proteins refers to the translation of the ion channel polypeptides from an ion channel gene sequence either from an endogenous gene or from nucleic acid introduced into a cell. The term “in situ” where used herein includes all these possibilities. Thus in situ methods may be performed in a suitably responsive cell line which expresses the ion channels. The cell line may be in tissue culture or may be, for example, a cell line xenograft in a non-human animal subject.

As used herein, the term “cell membrane” refers to a lipid bilayer surrounding a biological compartment, and encompasses an entire cell comprising such a membrane, or a portion of a cell.

For stable transfection of mammalian cells, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cell along with the gene of interest. Preferred selectable markers include those which confer resistance to drugs, such as G418, hygromycin and methotrexate. A nucleic acid encoding a selectable marker can be introduced into a host cell in the same vector as that encoding the ion channel proteins, or can be introduced in a separate vector. Cells stably transfected with the introduced nucleic acid can be identified by drug selection (e.g., cells that have incorporated the selectable marker gene will survive, while the other cells die).

It should be noted that expression of the ion channel proteins can also be controlled by any of a number of inducible promoters known in the art, such as a tetracycline responsive element, TRE. For example, the ion channel proteins can be selectively presented on the cell membrane by controlled expression using the Tet-on and Tet-off expression systems provided by Clontech (Gossen, M. and Bujard, H. Proc. Natl. Acad. Sci. USA 89: 5547-5551 (1992)). In the Tet-on system, gene expression is activated by the addition of a tetracycline derivative doxycycline (Dox), whereas in the Tet-off system, gene expression is turned on by the withdrawal of tetracyline (Tc) or Dox. Any other inducible mammalian gene expression system may also be used. Examples include systems using heat shock factors, steroid hormones, heavy metal ions, phorbol ester and interferons to conditionally expressing genes in mammalian cells.

The cell lines used in assays of the invention may be used to achieve transient expression of the ion channel proteins, or may be stably transfected with constructs that express an ion channel protein. Means to generate stably transformed cell lines are well known in the art, as well as described in U.S. Prov. Appl. No. 60/732,636, the disclosure of which is herein incorporated by reference, and such means may be used here. Examples of cells include, but are not limited to Chinese Hamster Ovary (CHO) cells, COS-7, HeLa, HEK 293, PC-12, and BAF.

The level of ion channel expression in a cell may be increased by introducing an ion channel nucleic acid into the cells or by causing or allowing expression from a heterologous nucleic acid encoding an ion channel. A cell may be used that endogenously expresses an ion channel without the introduction of heterologous genes. Such a cell may endogenously express sufficient levels of an ion channel for use in the methods of the invention, or may express only low levels of an ion channel which require supplementation as described herein.

The level of ion channel expression in a cell may also be increased by increasing the levels of expression of the endogenous gene. Endogenous gene activation techniques are known in the art and include, but are not limited to, the use of viral promoters (WO 93/09222; WO 94/12650 and WO 95/31560) and artificial transcription factors (Park et al. Nat. Biotech. 21:1208-1214 (2003).

The level of ion channel expression in a cell may be determined by techniques known in the art, including but not limited to, nucleic acid hybridization, polymerase chain reaction, RNase protection, dot blotting, immunocytochemistry and Western blotting. Alternatively, ion channel expression can be measured using a reporter gene system. Such systems, which include for example red or green fluorescent protein (see, e.g. Mistili and Spector, Nature Biotechnology 15:961-964 (1997), allow visualization of the reporter gene using standard techniques known to those of skill in the art, for example, fluorescence microscopy. Furthermore, the ability of TRPM5 to be activated by known positive modulating compounds, such as thrombin, may be determined following manipulation of the ion channel expressing cells.

Cells described herein may be cultured in any conventional nutrient media. The culture conditions, such as media, temperature, pH and the like, can be selected by the skilled artisan without undue experimentation. In general, principles, protocols, and practical techniques for maximizing the productivity of cell cultures can be found in “Mammalian Cell Biotechnology: a Practical Approach”, M. Butler, ed. JRL Press, (1991) and Sambrook et al, supra.

The cells can be grown in solution or on a solid support. The cells can be adherent or non-adherent. Solid supports include glass or plastic culture dishes, and plates having one compartment, or multiple compartments, e.g., multi-well plates. The multi-well vessels of the claimed invention may contain up to and a number equaling 96 wells. In another embodiment, the multi-well vessel comprises greater than 96 wells. In another embodiment, the multi-well vessel comprises 384 wells. In yet another embodiment, the multi-well vessel comprises 1536 wells.

The number of cells seeded into each well are preferably chosen so that the cells are at or near confluence, but not overgrown, when the assays are conducted, so that the signal-to-background ratio of the signal is increased.

### Ion Channel Activation

In order to observe ion channel activity, and evaluate whether a test compound can modulate activation, cells expressing the ion channels must be exposed to an activator. For the TRPM5 ion channel, intracellular calcium activators are used. TRPA1, TRPV1, TRPV3 and TRPM8 are activated by specific spicy ligands. Activation of TRPV1, for example, results in a rapid increase in intracellular Ca²⁺ levels (See, Cortright, D. N. and Szallasi, A. Eur. J. Biochem. 271:1814-1819 (2004)). There are many methods to activate intracellular calcium stores and many calcium activating agents are known in the art and include, but are not limited to thrombin, adenosine triphosphate (ATP), carbachol, and calcium ionophores (e.g. A23187). While nanomolar increases in calcium concentration ranges are required for TRPM5 channel activation, the concentration ranges useful for the claimed invention are known in the art, e.g., between 10⁻¹⁰ to 10⁻⁴ M for ATP. However, the precise concentration may vary depending on a variety of factors including cell type and time of incubation. The increased calcium concentration can be confirmed using calcium sensitive dyes, e.g., Fluo 3, Fluo 4, or FLIPR calcium 3 dye and single cell imaging techniques in conjunction with Fura2.

Test cells can also be incubated with lower doses of the calcium activating agents described above, such that a fluorescent response that is lower than the maximum achievable response is generated. Generally, the dose is referred to as the effect concentration or EC₂₀₋₃₀, which relates to the effect condition where the fluorescent intensity is 20-30% of the maximal response. As used herein, “EC” refers to effect condition, such that EC₂₀ refers to the effect condition where the fluorescent intensity is 20% of the maximal response is generated. Upon the addition of a second ion channel-specific activating compound, this low response will be increased to at, or near, maximal levels of activation.

### Detection of Ion Channel Activation

Movement of physiologically relevant substrates through ion channels can be traced by a variety of physical, optical, or chemical techniques (Stein, W. D., Transport and Diffusion Across Cell Membranes, 1986, Academic Press, Orlando, Fla.). Assays for modulators of ion channels include electrophysiological assays, cell-by-cell assays using microelectrodes (Wu, C.-F. et al., Neurosci 3(9):1888-99 (1983)), i.e., intracellular and patch clamp techniques (Neher, E. and Sakmann, B., Sci. Amer. 266:44-51 (1992)), and radioactive tracer ion techniques. Preferably, the effect of the candidate compound is determined by measuring the change in the cell membrane potential after the cell is exposed to the compound. This may be done, for example, using a fluorescent dye that emits fluorescence in response to changes in cell membrane potential and an optical reader to detect this fluorescence.

Optical methods using fluorescence detection are particularly suitable methods for high throughput screening of candidate compounds. Optical methods permit measurement of the entire course of ion flux in a single cell as well as in groups of cells. The advantages of monitoring transport by fluorescence techniques include the high level of sensitivity of these methods, temporal resolution, modest demand for biological material, lack of radioactivity, and the ability to continuously monitor ion transport to obtain kinetic information (Eidelman, O. et al., Biophys. Acta 988:319-334 (1989)). Present day optical readers detect fluorescence from multiple samples in a short time and can be automated. Fluorescence readouts are used widely both to monitor intracellular ion concentrations and to measure membrane potentials.

Voltage sensitive dyes that may be used in the assays and methods of the invention have been used to address cellular membrane potentials (Zochowski et al., Biol. Bull. 198:1-21 (2000)). Membrane potential dyes or voltage-sensitive dyes refer to molecules or combinations of molecules that enter depolarized cells, bind to intracellular proteins or membranes and exhibit enhanced fluorescence. These dyes can be used to detect changes in the activity of an ion channel such as TRPM5, expressed in a cell. Voltage-sensitive dyes include, but are not limited to, modified bisoxonol dyes, sodium dyes, potassium dyes and thorium dyes. The dyes enter cells and bind to intracellular proteins or membranes, therein exhibiting enhanced fluorescence and red spectral shifts (Epps et al., Chem. Phys. Lipids 69:137-150 (1994)). Increased depolarization results in more influx of the anionic dye and thus an increase in fluorescence.

In one embodiment, the membrane potential dyes are FMP dyes available from Molecular Devices (Catalog Nos. R8034, R8123). In other embodiments, suitable dyes could include dual wavelength FRET-based dyes such as DiSBAC2, DiSBAC3, and CC-2-DMPE (Invitrogen Cat. No. K1016). [Chemical Name Pacific Blue™ 1,2-ditetradecanoyl-sn-glycero-3-phosphoethanolamine, triethylammonium salt].

Calcium-sensitive fluorescent agents are also useful to detect changes in TRPA1 activity. Suitable types of calcium-sensitive fluorescent agents include Fluo3, Fluo4, Fluo5, Calcium Green, Calcium Orange, Calcium Yellow, Fura-2, Fura-4, Fura-5, Fura-6, Fura-FF, Fura Red, indo-1, indo-5, BTC (Molecular Probes, Eugene, Oreg.), and FLIPR Calcium3 wash-free dye (Molecular Devices, Sunnyvale Calif.). In one embodiment, the intracellular calcium dye is the FLIPR Calcium 3 dye available from Molecular Devices (Part Number: R8091). Additional calcium-sensitive fluorescent agents known to the skilled artisan are also suitable for use in the claimed assay. The calcium-sensitive fluorescent agents can be hydrophilic or hydrophobic.

Sodium-sensitive fluorescent agents are also useful to detect changes in TRPA1 activity. Suitable types of sodium-sensitive fluorescent agents include CoroNa™ Green, CoroNa™ Red chloride, SBFI, and Sodium Green™ (Molecular Probes, Eugene, Oreg.). Additional sodium-sensitive fluorescent agents known to the skilled artisan are also suitable for use in the claimed assay. The sodium-sensitive fluorescent agents can be hydrophilic or hydrophobic.

The voltage- or ion-sensitive fluorescent dyes are loaded into the cytoplasm by contacting the cells with a solution comprising a membrane-permeable derivative of the dye. However, the loading process may be facilitated where a more hydrophobic form of the dye is used. Thus, voltage- and ion-sensitive fluorescent dyes are known and available as hydrophobic acetoxymethyl esters, which are able to permeate cell membranes more readily than the unmodified dyes. As the acetoxymethyl ester form of the dye enters the cell, the ester group is removed by cytosolic esterases, thereby trapping the dye in the cytosol.

The ion channel-expressing cells of the assay are generally preloaded with the fluorescent dyes for 30-240 minutes prior to addition of candidate compounds. Preloading refers to the addition of the fluorescent dye for a period prior to candidate compound addition during which the dye enters the cell and binds to intracellular lipophilic moieties. Cells are typically treated with 1 to 10 μM buffered solutions of the dye for 20 to 60 minutes at 37° C. In some cases it is necessary to remove the dye solutions from the cells and add fresh assay buffer before proceeding with the assay.

Another method for testing ion channel activity is to measure changes in cell membrane potential using the patch-clamp technique. (Hamill et al., Nature 294:462-4 (1981)). In this technique, a cell is attached to an electrode containing a micropipette tip which directly measures the electrical conditions of the cell. This allows detailed biophysical characterization of changes in membrane potential in response to various stimuli. Thus, the patch-clamp technique can be used as a screening tool to identify compounds that modulate activity of ion channels.

Radiotracer ions have been used for biochemical and pharmacological investigations of channel-controlled ion translocation in cell preparations (Hosford, D. A. et al., Brain Res. 516:192-200 (1990)). In this method, the cells are exposed to a radioactive tracer ion and an activating ligand for a period of time, the cells are then washed, and counted for radioactive content. Radioactive isotopes are well known (Evans, E. A., Muramtsu, M. Radiotracer Techniques and Applications, M. Dekker, New York (1977)) and their uses have permitted detection of target substances with high sensitivity.

### Assay Detection

Detecting and recording alterations in the spectral characteristics of the dye in response to changes in membrane potential may be performed by any means known to those skilled in the art. As used herein, a “recording” refers to collecting and/or storing data obtained from processed fluorescent signals, such as are obtained in fluorescent imaging analysis.

In some embodiments, the assays of the present invention are performed on isolated cells using microscopic imaging to detect changes in spectral (i.e., fluorescent) properties. In other embodiments, the assay is performed in a multi-well format and spectral characteristics are determined using a microplate reader.

By “well” it is meant generally a bounded area within a container, which may be either discrete (e.g., to provide for an isolated sample) or in communication with one or more other bounded areas (e.g., to provide for fluid communication between one or more samples in a well). For example, cells grown on a substrate are normally contained within a well that may also contain culture medium for living cells. Substrates can comprise any suitable material, such as plastic, glass, and the like. Plastic is conventionally used for maintenance and/or growth of cells in vitro.

A “multi-well vessel”, as noted above, is an example of a substrate comprising more than one well in an array. Multi-well vessels useful in the invention can be of any of a variety of standard formats (e.g., plates having 2, 4, 6, 24, 96, 384, or 1536, etc., wells), but can also be in a non-standard format (e.g., plates having 3, 5, 7, etc., wells).

A suitable configuration for single cell imaging involves the use of a microscope equipped with a computer system. One example of such a configuration, ATTO's Attofluor® RatioVision® real-time digital fluorescence analyzer from Carl Zeiss, is a completely integrated work station for the analysis of fluorescent probes in living cells and prepared specimens (ATTO, Rockville, Md.). The system can observe ions either individually or simultaneously in combinations limited only by the optical properties of the probes in use. The standard imaging system is capable of performing multiple dye experiments such as FMP (for sodium) combined with GFP (for transfection) in the same cells over the same period of time. Ratio images and graphical data from multiple dyes are displayed online.

When the assays of the invention are performed in a multi-well format, a suitable device for detecting changes in spectral qualities of the dyes used is a multi-well microplate reader. Suitable devices are commercially available, for example, from Molecular Devices (FLEXstation® microplate reader and fluid transfer system or FLIPR1 system), from Hamamatsu (FDSS 6000) and the “VIPR” voltage ion probe reader (Aurora, Bioscience Corp. CA, USA). The FLIPR-Tetra™ is a second generation reader that provides real-time kinetic cell-based assays using up to 1536 simultaneous liquid transfer systems. All of these systems can be used with commercially available dyes such as FMP, which excites in the visible wavelength range.

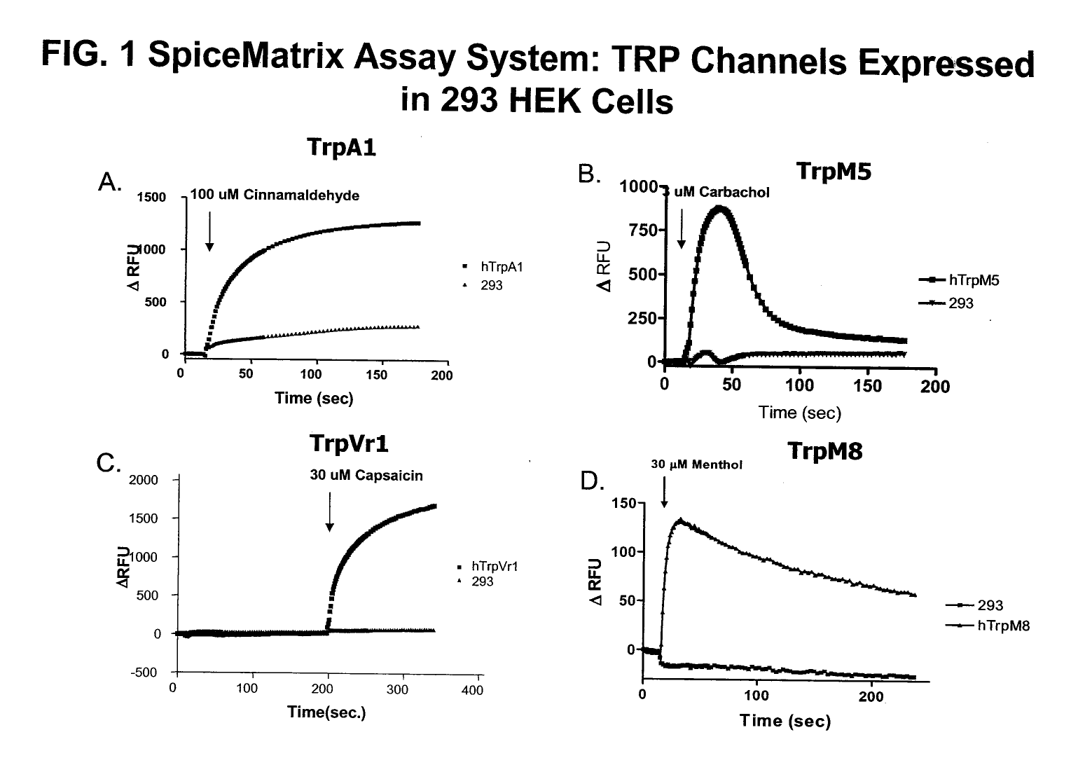

Using the FLIPR® system, the change in fluorescent intensity is monitored over time and is graphically displayed as shown, for example in FIG. 1. The addition of ion channel enhancing compounds causes an increase in fluorescence, while ion channel blocking compounds block this increase.

Several commercial fluorescence detectors are available that can inject liquid into a single well or simultaneously into multiple wells. These include, but are not limited to, the Molecular Devices FlexStation (eight wells), BMG NovoStar (two wells) and Aurora VIPR (eight wells). Typically, these instruments require 12 to 96 minutes to read a 96-well plate in flash luminescence or fluorescence mode (1 min/well). An alternative method is to inject the modulator into all sample wells at the same time and measure the luminescence in the whole plate by imaging with a charge-coupled device (CCD) camera, similar to the way that calcium responses are read by calcium-sensitive fluorescent dyes in the FLIPR®, FLIPR-384 or FLIPR-Tetra™ instruments. Other fluorescence imaging systems with integrated liquid handling are expected from other commercial suppliers such as the second generation LEADSEEKER from Amersham, the Perkin Elmer CellLux—Cellular Fluorescence Workstation and the Hamamatsu FDSS6000 System. These instruments can generally be configured to proper excitation and emission settings to read FMP dye (540ₑₓ±15 nm, 570ₑₘ±15 nm) and calcium dye (490ₑₓ±15 nm, 530ₑₘ±15 nm). The excitation/emission characteristics differ for each dye, therefore, the instruments are configured to detect the dye chosen for each assay.

The data generated by the optical detectors can be processed using a variety of computerized programs known in the art. For example, time-sequence files generated by the FLIPR® system can be processed using the data reduction package CeuticalSoft®. The CeuticalSoft® data package consists of: Kinetiture®, which views the kinetic traces, extracts FLIPR peak heights and marks outliers; Calcature®, which calculates normalized response (percent of control) for agonist assay (1st addition) and antagonist assay (2nd addition); and Curvature®, which calculates effective concentration for 50% activation (EC₅₀) and concentration for 50% inhibition (IC₅₀). The processed data can be stored in searchable databases, such as the Microsoft Access Database.

Finally, cheminformatics analysis can be performed using a 2D/3D cluster analysis of active structures within and between taste receptor (TRP) assays to group similar molecules. Models of compound structure versus comparative TRP channel activation can be created to assist in the potential identification of new TRP channel activating molecules.

### Candidate Compounds

Candidate compounds employed in the screening methods of this invention include for example, without limitation, synthetic organic compounds, chemical compounds, naturally occurring products, polypeptides and peptides, nucleic acids, etc.

Essentially any chemical compound can be used as a potential modulator or ligand in the assays of the invention. Most often compounds dissolved in aqueous or organic (especially dimethyl sulfoxide- or DMSO-based) solutions are used. The assays are designed to screen large chemical libraries by automating the assay steps. The compounds are provided from any convenient source to the cells. The assays are typically run in parallel (e.g., in microtiter formats on microtiter plates in robotic assays with different test compounds in different wells on the same plate). It will be appreciated that there are many suppliers of chemical compounds, including ChemDiv (San Diego, Calif.), Sigma-Aldrich (St. Louis, Mo.), Fluka Chemika-Biochemica-Analytika (Buchs Switzerland) and the like.

“Modulating” as used herein includes any effect on the functional activity of the ion channels. This includes blocking or inhibiting the activity of the channel in the presence of, or in response to, an appropriate stimulator. Alternatively, modulators may enhance the activity of the channel. “Enhance” as used herein, includes any increase in the functional activity of the ion channels.

In one embodiment, the high throughput screening methods involve providing a small organic molecule or peptide library containing a large number of potential ion channel modulators. Such “chemical libraries” are then screened in one or more assays, as described herein, to identify those library members (particular chemical species or subclasses) that display a desired characteristic activity. The compounds thus identified can serve as conventional “lead compounds” or can themselves be used as potential or actual products.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175; Furka Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al, J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14:309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky.; Symphony, Rainin, Woburn, Mass.; 433A Applied Biosystems, Foster City, Calif.; 9050 Plus, Millipore, Bedford, Mass.). In addition, numerous combinatorial libraries are themselves commercially available (see, e.g., ComGenex, Princeton, N.J.; Asinex, Moscow, Russia; Tripos, Inc., St. Louis, Mo.; ChemStar, Ltd, Moscow, Russia; 3D Pharmaceuticals, Exton, Pa.; Martek Biosciences, Columbia, Md.; etc.).

Candidate agents, compounds, drugs, and the like encompass numerous chemical classes, though typically they are organic molecules, preferably small organic compounds having a molecular weight of more than 100 and less than about 10,000 daltons, preferably, less than about 2000 to 5000 daltons. Candidate compounds may comprise functional groups necessary for structural interaction with proteins, particularly hydrogen bonding, and typically include at least an amine, carbonyl, hydroxyl or carboxyl group, preferably at least two of the functional chemical groups. The candidate compounds may comprise cyclical carbon or heterocyclic structures, and/or aromatic or polyaromatic structures substituted with one or more of the above functional groups. Candidate compounds are also found among biomolecules including peptides, saccharides, fatty acids, steroids, purines, pyrimidines, derivatives, structural analogs or combinations thereof.

A variety of other reagents may be included in the screening assay according to the present invention. Such reagents include, but are not limited to, salts, solvents, neutral proteins, e.g. albumin, detergents, etc., which may be used to facilitate optimal protein-protein binding and/or to reduce non-specific or background interactions. Examples of solvents include, but are not limited to, dimethyl sulfoxide (DMSO), ethanol and acetone, and are generally used at a concentration of less than or equal to 1% (v/v) of the total assay volume. In addition, reagents that otherwise improve the efficiency of the assay, such as protease inhibitors, anti-microbial agents, etc. may be used. Further, the mixture of components in the method may be added in any order that provides for the requisite binding.

The compounds identified using the disclosed assay are potentially useful as ingredients or flavorants in ingestible compositions, i.e., foods and beverages as wells as orally administered medicinals. Compounds that modulate taste perception can be used alone or in combination as flavorants in foods or beverages. The amount of such compound(s) will be an amount that yields the desired degree of modulated taste perception of which starting concentrations may generally be between 0.1 and 1000 μM.

### EXAMPLES

### Example 1

### SpiceMatrix Analysis of Taste Compounds

As described in greater detail below, HEK 293 cells, transiently transfected with plasmid bearing the genes encoding the various ion channels, were used to develop SpiceMatrix fingerprint assay. Indirect measurement of the changes in ion concentrations within the HEK 293 cells were made using a FMP dye and stimulation of the cells using calcium activating agents. Described below are the conditions for screening using TRPM5. Screening conditions for other TRP ion channels were similar to those described unless otherwise indicated.

### TRPM5

### Plasmid Construction

First strand cDNA was synthesized by Thermoscript RT-PCR System (Invitrogen) from human small intestine poly A+ RNA (BD Biosciences) and the full length hTRPM5 was amplified by PCR using GC Melt (BD Biosciences). The product was PCR purified by Pure Link PCR Purification (Invitrogen) and inserted into a vector using the TOPO TA Cloning Kit (Invitrogen). After sequencing, 6 mutations were found and the mutations were corrected using the Quick Change Multi Site Directed Mutagenesis Kit (Stratagene) in 2 rounds. Three mutations were corrected in each round. The full length TRPM5 was excised from the TOPO TA vector using the EcoRI and NotI restriction enzymes and ligated in the pENTR 3C vector, which had also been digested with EcoRI and NotI. The insert and vector bands were gel extracted and purified using the SNAP Gel Purification Kit (Invitrogen). Finally, LR Recombination Reaction (Invitrogen) was used to insert the entry clone into destination vectors of interest (e.g., pT-Rex-DEST 30, pcDNA-DEST 53, pcDNA 3.2/v5-DEST and pcDNA 6.2/V5-DEST).

### Transfection

1.0×10⁶ HEK 293 cells (ATCC) were plated in each well of a 6-well tissue culture dish overnight. The following day, cells were transfected with 4 μg of a pcDNA3.2 vector containing TRPM5 cDNA and 8 μl of Lipofectamine 2000 (Invitrogen), according to the manufacturer's protocol, and incubated overnight. The following day, transfected cells were trypsinized and seeded into 96-well black, clear bottom, poly-D-lysine plates (Corning) at a density of 70,000 cells/well in a 100 μl volume and incubated in a 37° C./5% CO₂ incubator overnight.

### Membrane Potential Assay

Once the expression of TRPM5 was confirmed in the HEK cells, 100 μl of the Blue or Red FMP dye (Molecular Devices) was added to each well of plates seeded with the transiently transfected cells. The plate was then incubated in a 37° C./5% CO₂ incubator for 1 hour. The plate was read in a FLEXStation microplate reader (Molecular Devices) with an excitation of 530 nm and an emission of 565 nm. The fluorescence was monitored for 3 minutes upon exposure of the cells to a calcium activating agent (carbachol, thrombin peptide or ATP).

For screening of taste compounds, sample dilution sets (four 384 sample plates) were tested in 5, dye-loaded, cell lines to yield 20 assay plates for data collection. One cell plate, a sample plate, and an agonist sample source plate were placed in the FLIPR. To identify samples with agonist activity, 10 μl of samples or standards were added to the cell plate, and sample agonist response fluorescent readings taken for 3 minutes. To identify samples with antagonist activity, agonist, e.g. 100 μM cinnamaldehyde for TRPA1, was added to all wells and agonist response fluorescent readings were taken for 2 minutes. Sample that block this response were nominally antagonists.

### Results

Demonstration of ion channel responses is shown in FIG. 1. TRPA1, TRPM5, TRPV1 and TRPM8 transfected cells were loaded with FMP dye and then treated with cinnamaldehyde (FIG. 1A), carbachol (FIG. 1B), capsaicin (FIG. 1C) and menthol (FIG. 1D) and monitored for an increase in cellular fluorescence in the FLIPR®. All four agents generated a strong spike in relative fluorescence following agonist addition.

### SpiceMatrix Generation

The reactivity of 68 known taste compounds on TRPA1, TRPV1, TRPM8 and TRPM5 was determined using the above-described fluorescence assays. As shown in FIG. 2A, cinnamaldehyde and (−) menthol showed the greatest stimulation of TRPA1 and varying degrees of stimulation of the other ion channels; while, gingerol showed the highest degree of stimulation to TRPV1. A SpiceMatrix is shown for each of the compounds which reflects their effect on the activity of the TRPA1, TRPV1, TRPM8 and TRPM5 ion channels. The reactivity profiles for each compound was validated using dose response studies (FIGS. 2B-2C). The reactivity profiles of a 23-member subset of the 68 compounds described above is shown in FIG. 3.

### Example 2

### SpiceMatrix Analysis of Odor Compounds

SpiceMatrix analysis is performed on 100 odor compounds. To characterize the potential taste properties of pure odor molecules in 4 specific TRP ion channels important in taste responses: TRPA1 (cinnamaldehyde responsive), TRPM8 (menthol), TRPV1 (capsaicin) and TRPV3 (vanillin). A 5th cell line, nontransfected parental, are included for control purposes.

The 100 pure compounds are tested using the SpiceMatrix analysis in the FLIPR (Fluorometric Imaging Plate Reader) optical detector. Both agonist and antagonist activities of samples are tested in duplicate in a 5 point curve covering a concentration range of 1 μM to 500 μM (10 points/compound/assay). The full assays are run twice (separate days) to strengthen the validity of the data.

### Sample Preparation

All samples are diluted in 100% DMSO in 5 fold steps. 20 μl aliquots are diluted 1:5 with 100% DMSO achieving 50, 10, 2, 0.4 and 0.08 mM stock solutions in a 96 well plate. The above 100% DMSO solutions are then diluted 1:20 into a physiologic buffer immediately prior to assay on the FLIPR which involves another 1:5 dilution. Samples are reformatted into a bar-coded 384 well polypropylene sample plate. Assay standards, (positive and negative controls and an agonist dose response curve) are added to the sample plate.

### Preparation of Cells for Screening

HEK 293 cell suspensions, 20 μl, containing ˜10,000 cells are seeded in clear bottom 384 well FLIPR imaging plates. Typically sets of 6-7 plates (2-3 are extra) are made for each of the cell lines for each of the 4 TRP channels and control line. Plated cells are kept overnight in a CO₂ incubator to allow for cell attachment to the bottom of the plate. The next day, Membrane Potential Dye, 20 μl, is added, and the cells are put back in the incubator for an hour to allow for dye uptake. Cell plates are removed from the incubator and put at room temperature for 30 minutes for temperature equilibration.

### FLIPR Data Collection

Eighty-eight sample dilution sets (four 384 sample plates) are typically tested in 5, dye-loaded, cell lines to yield 20 assay plates for data collection. One cell plate, a sample plate, and an agonist sample source plate are placed in the FLIPR. Plates are bar-coded to generate the output data file identifications. To identify samples with agonist activity, the assay is started, 10 μl of samples or standards are added to the cell plate, and sample agonist response fluorescent readings taken for 3 minutes. To identify samples with antagonist activity, agonist, e.g. 100 μM cinnamaldehyde for TRPA1, is added to all wells and agonist response fluorescent readings are taken for 2 minutes. Sample that block this response are nominally antagonists. Cell and sample plates are then removed and new cell and compound plates are put in FLIPR. This assay cycle continues until all 20 assays are completed. Markedly defective plates are normally spotted during the run and are retreated in the assay cycle.

### Data Analysis

FLIPR data files, or so-called time sequence files, for each assay are processed in CeuticalSoft® FLIPR assay data reduction package consisting of: Kinetiture®, which views the kinetic traces, extracts FLIPR peak heights and marks outliers; Calcature®, which calculates normalized response (percent of control) for agonist assay (1st addition) and antagonist assay (2nd addition); and Curvature®, which calculates the effective concentration for 50% activation (EC₅₀) and the concentration for 50% inhibition (IC₅₀).

The data generated can be used to manipulate the taste perception of a given compound. By manipulating the activity of compounds to the ion channels, the taste perception of a compound can be altered in the desired manner. Useful examples in which manipulation may be beneficial include, but are not limited to, medicines in which the active ingredients produce undesirable tastes or for enhancing pleasurable tastes in food products.

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents. All publications, patents and patent applications cited herein are incorporated by reference in their entirety into the disclosure.

What is claimed is:

1. A method for generating a reactivity profile for compounds that affect taste comprising:
(a) contacting said compound with at least two groups of isolated test cells expressing a transient receptor potential (TRP) ion channel, wherein each group of test cells expresses a different recombinant TRP ion channel;
(b) measuring the activity of the test cells of step (a) in the presence of the compound;
(c) comparing the measured activity in step (b) to the activity of the test cells which do not express a TRP ion channel in the presence of the compound to determine the extent of TRP modulation; and
(d) generating a reactivity profile of the at least two TRP ion channels.

2. The method of claim 1, wherein at least two of the TRP ion channels are selected from the group consisting of: TRPA1, TRPV1, TRPV3, TRPM8 and TRPM5.

3. The method of claim 2, wherein three or more TRP ion channels are used.

4. The method of claim 2, wherein four or more TRP ion channels are used.

5. The method of claim 1, wherein the activity is determined by measuring the fluorescent intensity of the cell.

6. The method of claim 1, wherein the activity is determined in a high throughput assay.

7. The method of claim 1, wherein the cells are located in a multi-well vessel.

8. The method of claim 7, wherein the multi-well vessel comprises up to 96 wells.

9. The method of claim 7, wherein the multi-well vessel comprises greater than 96 wells.

10. The method of claim 7, wherein the multi-well vessel comprises 384 wells.

11. The method of claim 7, wherein the multi-well vessel comprises 1536 wells.

12. The method of claim 1, wherein the test cells are selected from the group consisting of: HEK-293, Hela, Chinese Hamster Ovary, COS, RBL and PC12.

13. The method of claim 12, wherein the test cells are HEK-293 cells.

14. The method of claim 5, wherein the fluorescent intensity is measured using a membrane potential fluorescent dye.

15. The method of claim 14, wherein the membrane potential fluorescent dye is a Fluorescent Imaging Plate Reader Membrane Potential (FMP) dye.

16. The method of claim 14, wherein the fluorescent intensity is measured using a calcium dye.

17. The method of claim 5, wherein the fluorescent intensity is measured using an optical detector.

18. The method of claim 17, wherein the optical detector is selected from the group consisting of: Fluorescent Imaging Plate Reader (FLIPR®), FLEXStation, Voltage/Ion Probe Reader (VIPR), fluorescent microscope and charge-coupled device (CCD) camera, and Pathway HT.

19. The method of claim 18, wherein the optical detector is a FLIPR®.

20. A method of manipulating the taste profile of a compound comprising:
(a) contacting said compound with at least two groups of isolated test cells expressing a TRP ion channel, wherein each group of test cells expresses a different recombinant TRP ion channel;
(b) measuring the activity of the test cells of step (a) in the presence of the compound;
(c) comparing the measured activity in step (b) to the activity of the test cells which do not express a TRP ion channel in the presence of the compound to determine the extent of TRP modulation;
(d) generating a reactivity profile of the at least two TRP ion channels; and
(e) altering the reactivity of said compound with said TRP ion channels.

21. A method for identifying novel taste compounds comprising:
(a) determining the reactivity of known taste compounds to at least two groups of isolated test cells expressing a TRP ion channel, wherein each group of test cells expresses a different recombinant TRP ion channel;
(b) contacting at least two different groups of isolated test cells expressing a TRP ion channel with a potential taste compound, wherein the test cells express the same recombinant TRP ion channels as in step (a);
(c) measuring the activity of the test cells of step (b) in the presence of the potential taste compound;
(d) comparing the measured activity to the activity of test cells that do not express a TRP ion channel to determine the extent of TRP modulation;
(e) comparing the reactivity of known taste compounds of step (a) to the reactivity of the potential taste compound to the at least two different recombinant TRP ion channels; and
(f) selecting one or more taste compounds that display a similar TRP ion channel reactivity pattern to known taste compounds.
